(19) United States
(12) Patent Application Publication
(10) Pub. No.: US 2005/0143299 A1
(43) Pub. Date: Jun. 30, 2005

(54) Use of relaxin as adjuvant in the differentiation of stem cells for the reconstruction of tissues

(76) Inventors: Bernardo Bigazzi, Firenze (IT); Mario Bigazzi, Firenze (IT)

Correspondence Address: MCGLEW & TUTTLE, PC, P.O. BOX 9227, SCARBOROUGH STATION, SCARBOROUGH, NY, 10510-9227, US

(21) Appl. No.: 10/873,973

(22) Filed: Jun. 22, 2004

(30) Foreign Application Priority Data
Dec. 24, 2003 (IT) FI2003A000328

(51) Int. Cl. A61K 38/17; C12N 5/08

(52) U.S. Cl. 514/12; 435/366; 435/368; 424/93.7

(57) ABSTRACT

On the basis of the experimental results it has emerged that relaxin has a pro-differentiating effect on stem cells. It is therefore suggested the use of this hormone as principal component of a drug for the treatment of all those situations that find or will find benefit from the use of stem cells for the reconstruction of tissues damaged by traumatic events or by ischemic-inflammatory-degenerative diseases. It is also suggested the use of relaxin for the treatment of syndromes deriving from the missing activation of stem cells during fetal development (at subsequent somatic-functional maturation), such as for example hypogonotropic hypogonadism with anosmia (Kallman's syndrome).

### TECHNICAL FIELD

The present invention relates in a broad sense to the activation and/or differentiation of stem cells so that it would be possible to achieve the purpose of their use or of their activation in situ both for the regeneration of tissues damaged or subject to ischemic-inflammatory degenerative diseases and for the development or restoration of organic functions produced also by lack, deficiency of development or differentiation of cells during fetal life and subsequent somatic-functional maturation.

In particular, but not exclusively, the present invention relates to the reconstruction of cardiac tissue of nervous tissue, of muscular tissue, osteocartilagineous tissue, hepatic tissue and of any other organ that might require a reconstructive therapy in consequence of lesions deriving from degenerative diseases, from traumatic events, from surgical interventions or other.

### BACKGROUND OF THE INVENTION

Stem cells currently represent one of the fields of greater interest in biomedical research. This term is used to identify undifferentiated cells, for the most part originating from the first phases of embryonal development (totipotent or pluripotent stem cells, with high differentiative potential) or from adult tissues containing physiologically undifferentiated elements such as the hematopoietic bone marrow (multipotent stem, with differentiative potential limited to some cellular stipes).

Methods for isolating or propagating selectively animal stem cells are described in EP-A-0695351.

The studies on the stem cells hold an interest of absolute clinical relief, in that they open new fields of therapeutic application for all those diseases characterized by the loss of key cells that the organism would not be able to replace spontaneously, such as for example neurodegenerative diseases and ischemic diseases of the myocardium.

It should be emphasized that there are already consolidated clinical applications of the stem cells, which to date permit the healing of many cases of leukemia and of lymphoma by means of transplantation of the multipotent hematopoietic stem cells that replace in toto the neoplastic marrow.

Much more complex and still far from a direct clinical application is the research concerning the possibility of replacing with stem cells differentiated elements of the nerve tissue or of the cardiac muscle. Potentially the possibility exists of directing undifferentiated precursors towards a cellular phenotype for example neuronal or cardiac muscular, by means of suitable cocktails of growth factors and cytokines in culture in vitro, in tissues and organs isolated or in experimental animals.

The principal limit that still interposes itself between these preliminary results and an efficacious therapeutic application consists in the scarcity of the new cells that succeed in integrating themselves correctly and efficiently in the host tissue, very often obstructed in that by the altered conditions of the tissue damaged to be repaired, seat of inflammation, fibroses or repair glioses. They are, therefore, of extreme urgency the studies capable of identifying the stimuli by means of which to induce the differentiation of the stem cells towards a given phenotype or potentiate their integration in the tissue to be repaired.

The use of stem cells in the repair of the tissue of the myocardium damaged by infarction has been at the centre of numerous studies.

In Hagege A. A., Vilquin J. T., Bruneval P. and Menaschè P. (2001) Regeneration of the myocardium. A role in the treatment of ischemic heart disease? Hypertension, 2001, No. 38: 1413-1415, is described the possibility of reconstructing cardiac tissue by means of injection of fetal cardiomyocytes or myoblasts.

Chiu R. C. J., Zibaitis A. and Kao R. (1995) Cellular cardiomyoplasty: myocardial regeneration with satellite cell implantation, Ann. Thorac. Surg., 1995, 60: 12-18 describe the possibility of executing cellular cardiomyoplasty treatments by means of implantation of skeletal myoblasts in the myocardium. Similar studies are described in Murry C. E., Wiseman R. W., Schwartz S. M. and Hauschka S. D. (1996) Skeletal myoblasts transplantation for repair of myocardial necrosis, J. Clin. Invest., 1996, No. 98: 2512-2523.

Various studies have also been carried out for investigating the possibility of introducing the myoblasts in the damaged cardiac tissue by means of administration by the arterial route. See in this connection among others Robinson S. W., Cho P. W., Levitsky H. M., Olson J. L., Hruban R. H., Acker M. A. and Kessler P. D. Arterial delivery of genetically labelled skeletal myoblasts to the murine heart: long-term survival and phenotypic modification of implanted myoblasts, in Cell. Transplantation, 1996, Vol. 5: 77-91; and also Suzuki K., Brand N. J., Smolensky R. T., Jayakumar J., Martuza B. and Yacoub M. H. Development of a novel method for cell transplantation through the coronary artery, in Circulation, 2000, Vol. 102: 111-359-364.

The possibility of restoring the morphological continuity and the normal cardiac activity in the case of infarction, becomes particularly relevant in consideration of the fact that the myocardial tissue is a perennial tissue that does not possess its own regenerative capacity, being constituted of cells highly differentiated for the contractile function, the cardiomyocytes, with scant or absent proliferative capacity. The various experimental attempts capable of promoting the transformation of non-myogenic elements in contractile cells as well as for inducing the cardiomyocytes to resume the cellular cycle have proved up to now completely fruitless.

As reported also in the literature cited above, more promising appear, in contrast, the attempts with cells already predetermined towards a muscular phenotype. The cellular types used for this purpose are represented substantially by fetal cardiomyocytes and by undifferentiated skeletal muscular cells (myoblasts), normally present in the skeletal muscular tissue for guaranteeing the tissue regeneration following damage. In particular, the fetal cardiomyocytes can easily be cultivated and expanded in vitro but, from the clinical point of view, their use in the myocardial regeneration has raised some important problems, both technical and ethical, connected with the isolation of the cells from embryonal hearts and with the onset of immunological phenomena.

Vice versa, the use of autologous myoblastic cells (i.e. originating from the same patient) has been proving to be a very promising technique in this direction, both because it does not require the immunosuppressant treatment of the recipient, and because it is a question of cells highly resistant to ischemia. In favor of the use of these cells in the myocardial regeneration, there are also some recent results that have demonstrated that the myoblasts present, at least in the first phases of their differentiation, some morpho-functional characteristics entirely similar to those of the cardiomyocytes. They are, in fact, joined by specialized intercellular junctions—the gap junctions—which form a kind of transmembrane channel between the adjacent cells, permitting their metabolic and ionic coupling. Analogously to the gap junctions present in the myocardium, also those present in the myoblasts are formed by the connexin 43 (Cx43) protein.

It seems established that the communicating junctions play an important role during the process of differentiation of the myoblasts, facilitating their fusion in multinucleated elements, the myotubes. In the myocardium, the protein Cx43 is concentrated in the scalariform striae that join the cardiomyocytes at the level of the cellular extremities, where it becomes necessary for the transmission of the electrical impulse between adjacent cells and for the coordination of the contractile activity. It is however important to emphasize that such junctions are present in the myoblasts in a very early differentiation stage and decrease in the subsequent phases of maturation (myotubes) and are practically absent in the mature skeletal muscular fibers.

Therefore, with differentiation completed, the two muscular tissues, the skeletal and the cardiac, become profoundly different not only in morphological characteristics but also mechanical, the skeletal fibers, in contrast to the cardiomyocytes, being electrically isolated from one another. Studies in vivo carried out on experimental animals in which infarction had been induced experimentally before inoculation in the infarcted site of the myoblastic cells, have revealed that these cells are effectively capable of colonizing the necrotic area and of differentiating in situ in myotubes, giving rise to the formation of new muscular tissue which limits considerably the extent of the fibrotic area. Moreover, the neoformed skeletal muscular tissue contracts if stimulated electrically, giving rise to a significant increase of the ventricular performance (i.e. increases the ejection fraction). Said tissue is composed of fibers that develop morphological and functional characteristics intermediate between the skeletal tissue of origin and the cardiac tissue in which their differentiation occurs. The possibility that the cardiac micro-environment is able to influence and modify the differentiation program of the inoculated myoblasts has been further confirmed by the presence in the neo-tissue of muscular fibers with scalariform striae and nuclei localized centrally, by the anomalous expression in these fibers of factors involved in the intracellular homeostasis of Ca²⁺ typical of the cardiomyocytes, as well as by their conversion from fast fibers to fibers with slow contraction, more resistant to fatigue.

However, despite the recognition of the possible use of the myoblasts for the reconstruction of the infarcted myocardium, the experiments conducted up to now inoculating myoblasts in the infarcted heart or transporting them by the arterial route, among which those reported in the literature cited in the foregoing, have given results little encouraging. This because of the fact that the myoblasts inoculated do indeed differentiate in situ in muscular elements, but of skeletal type and not integrated functionally with the surviving cardiomyocytes.

### OBJECTS AND SUMMARY OF THE INVENTION

The invention has the purpose of finding an agent having pro-differentiating effects on the stem cells useful in reconstructive therapies, both of the myocardium, and of the tissues of the nervous system and also of other tissues in general, and especially of those tissues that possess reduced capacity for repairing the losses undergone by damage deriving from degenerative diseases or also from traumatic events.

In particular, the invention aims to identify agents capable of stimulating the differentiation of stem cells towards a determined phenotype or capable of potentiating the integration of them in the surviving tissue that we wish to repair.

It has surprisingly been discovered that relaxin (also indicated as RLX in the following), a typical hormone of pregnancy and that is present in the blood that flows to the fetus via the umbilical vein and possesses receptors in various bodily compartments, including the brain and the heart, has a pro-differentiating effect on the stem cells. The invention is therefore based on the use of relaxin or of its derivatives as modulating agent of the differentiation of the stem cells and especially on the myoblasts and on the neuroblasts.

Relaxin is a hormone that facilitates the relaxation of the pelvic ligaments in mammals. Its existence has been observed since 1926. A method for the extraction of human RLX is described for example in U.S. Pat. No. 4,267,101.

Methods of production of relaxin are described in U.S. Pat. No. 4,758,516, U.S. Pat. No. 4,835,251, U.S. Pat. No. 5,023,321 and in other publications recalled there. Further methods for the production of relaxin are described in U.S. Pat. No. 5,464,756.

Recently, this polypeptide hormone is at the centre of numerous studies and researches in consideration of the various therapeutic effects that it seems to possess. In U.S. Pat. No. 5,166,191 is described the use of relaxin as therapeutic agent for cardiac insufficiency.

In U.S. Pat. No. 5,952,296 are described uses of relaxin based on its effects of stimulation of the synthesis and of the release of NO. This effect is utilized for the treatment of pathologies connected with the circulatory system.

In U.S. Pat. No. 5,451,572 and in U.S. Pat. No. 5,945,402 are described methods for obtaining various types of pharmaceutical compositions containing a therapeutically effective quantity of human relaxin.

U.S. Pat. No. 5,811,395 and U.S. Pat. No. 5,911,997 and U.S. Pat. No. 6,200,953 describe relaxin, derivatives and analogs of relaxin and methods for the preparation of pharmaceutical compositions for the use in the therapy of scleroderma, of cardiovascular, neurodegenerative and neurological diseases and of other dysfunctions or pathological situations.

The possibility of employing relaxin in combination with estrogens for the symptomatic treatment of the loss of memory connected with Alzheimer's disease is described in U.S. Pat. No. 6,251,863. In this document are in general discussed the possibilities of treating with relaxin various pathological situations connected with aging. In particular are suggested also therapeutic methods of treatment of other dysfunctions, such as in particular the osteodegenerative dysfunctions by means of combined administration of relaxin and glusocamine sulfate.

In U.S. Pat. No. 6,048,544 is described a method for treating involuntary muscular dysfunctions. The method of treatment envisages the administration of relaxin in an emollient vehicle for cutaneous applications.

In U.S. Pat. No. 6,075,005 is described the use of relaxin in combination with an anti-androgenic agent, such as finasteride, estrogen, minoxidil. Methods are also described for the treatment of androgenic alopecia by means of the combination of the aforesaid components.

U.S. Pat. No. 6,211,147 describes methods for the promotion of angiogenesis by means of the administration of human recombinant relaxin.

As is clear from the patent literature cited above and from other numerous bibliographical sources cited in said publications, many uses of relaxin, based on various of its effects are under investigation and suggested for the therapeutic or prophylactic treatment of various pathological or degenerative situations. However, there has never been identified or recognized an effect of this hormone on the development of the stem cells.

In the context of the present description and of the appended claims, the term “relaxin” (or briefly RLX) will refer in general to “relaxin”, “human relaxin”, “native relaxin”, “synthetic relaxin” or analogs of relaxin. The term “relaxin” also encompasses porcine relaxin or obtained from other mammals, as well as relaxin produced by means of recombinant techniques and in particular human recombinant relaxin. Said term comprises, moreover, prbrelaxin, preprorelaxin, the derivatives and the analogs of relaxin, peptides and in general active agents or fragments of relaxin having the activity of relaxin, and in particular, capable of binding to the receptors of relaxin or of interacting with them. The term “relaxin” comprises in a specific manner variants of relaxin obtained for example by means of addition, substitution or removal of one or more of the components of relaxin. Examples of techniques for obtaining relaxin, its derivatives or analogs as well as the sequences that describe them, are described in the literature cited previously.

According to a first aspect, the invention relates to the therapeutic application of RLX in the post-infarction repair of the injured cardiac tissue, to promote the recolonization of the infarcted myocardium with cells with contractile properties. In substance, a first possibility of use of the hormone RLX is in the therapy of the post-infarction complications known as cellular cardiomyoplasty (CCM). This therapeutic strategy consists of promoting the regeneration of the injured myocardial area by intracoronary or intratissue injection of immature cells that differentiate in loco.

Relaxin can be injected to the patient together with the stem cells (whether these are myoblasts or fetal cardiomyocytes or in general totipotent, multipotent or pluripotent stem cells of various origin). Furthermore, it can also be used for cultivating in vitro the cells to be injected subsequently to the patient.

The efficacy of RLX as pro-differentiating factor on the stem cells is not limited to the myoblasts and to the fetal cardiomyocytes. Similar effects have been found on other types of stem cells and especially on neuroblasts. The possibility of directing these precursors to develop as far as to form adult nerve cells opens the way to the efficacious therapeutic use of these stem cells in the reconstructive therapy of nerve tissues damaged by neurodegenerative diseases or also by traumatic events, such as for example in nerve injuries of the spinal cord.

More generally, the experimental results referred to above, and that will be presented in greater detail in the following, allow to conclude that relaxin constitutes in general a pro-differentiating factor applicable to various types of stem cells for directing them towards a desired mature phenotype.

Therefore, a first object of the present invention is the use of relaxin for the production of a drug having a pro-differentiating effect on stem cells. Specifically, but not exclusively, an object of the invention is the use of relaxin for inducing a pro-differentiating effect on totipotent, pluripotent or multipotent stem cells, for example the myoblasts or the stem cardiomyocytes, for directing them to develop into mature cells that are able to integrate in the tissue of the myocardium.

According to a different aspect, the invention relates to the use of relaxin in combination with stem cells, especially nerve stem cells, and in particular in combination with neuroblasts, for directing their development into mature nerve cells that can integrate into the damaged nervous tissue.

An object of the invention is also the use of relaxin as pro-differentiating agent on stem cells, especially but not exclusively of the nervous or cardiac type.

A further example of possible use of RLX is represented by the osteo-cartilagineous cells.

Osseous tissue and cartilagineous tissue are liable to destruction and damage with loss of their function through inflammation, injuries, degeneration etc. In particular the cartilagineous tissue is not easily replaced in the adult organism producing serious functional limitations (e.g. condylitis-arthrosis).

Attempts to produce in vitro cellular cultures both for studying the synthesis of bone or of cartilage and for replacing parts of missing or damaged cartilage, are largely impeded by the fact that in the so-called “primary cultures” the cellular populations (in vitro) are in heterogeneous form and change genotype over time, with difficulties in producing and maintaining specific differentiations.

Moreover the cells in culture display low vitality and tendency to maturation.

It is known that joint cartilage has scant capacity for repair. In some experimental models a partial repair of the altered cartilage in subjects with osteoarthrosis was obtained with implantation of epiphyseal cartilage of young animals (Aston J. E. 1986—Bentley G. 1971). However, in said experiments the quantity of matrix produced was scant and the cartilagineous cells transplanted appeared surrounded by inflammatory cells and by fibrous tissue.

Preliminary experiments of the present inventors have supplied data that would confirm the stimulating effect of RLX on the development and on the differentiation of the mesenchymal blast cells towards the mature cells of the osseous and cartilagineous tissue, with the induction of the synthesis of specific substances (markers) of said cells and detection in the cells, in the electron microscope, of specific organelles involved in the production of the extracellular matrix (in particular collagen (I and II) and proteoglycans).

The ability of RLX to direct the undifferentiated mesenchymal cells (blasts) or of other tissue towards cartilagineous and osseous cells might have great clinical application in the repair of joint damage, in particular by means of the reconstruction of the damaged or missing cartilagineous tissue both by injection in loco of cells activated beforehand by RLX, and by stimulation of primitive mesenchymal cells (blasts) to differentiate and replace the damaged tissue with relaxin administered by the local or general route.

The data on the effects of RLX on the myoblasts and cardiomyocytes also suggest a use of RLX in the repair of injuries of muscles or tendons of other origin. For example it is possible to hypothesize its application in the healing or replacement of tissue damage in skeletal muscles and of the associated tendons through activation of blasts present in the injured tissue or injected in loco.

The activation and differentiation of said cells can take place with administration of RLX both local and parenteral (in situ or by the general route) and through the pre-activation “in vitro” of blast cells injected then in the site of the injury.

All these possibilities can also coexist.

A further and important example of use is represented by the potential of RLX to produce rapidly the maturation and organization of the tissue in organs affected by inflammatory-ischemic degenerative lesions in which cellular regeneration takes place but in a chaotic and disorganized manner.

The best example of this type is represented by the hepatic cellular proliferation in the course of chronic hepatitis, with production of upheaval of the architecture and hepatic function which develops with time towards the production of true and proper tumors.

Still in the field of hepatic pathology RLX might promote and facilitate hepatic regeneration after hepatic resections (e.g. for removal of tumors). In this case a local, general or oral administration or via the portal circulation can be envisaged, using various pharmaceutical formulations, such as solutions for parenteral use, tablets, suppositories.

There may be similar reconstructive effects on tissues or organs of other nature and in consequence of damaging or degenerative events of varying nature, including the surgical interventions of resection and traumatic events.

All these effects can be obtained with RLX both administered on its own, and in combination with other biological or pharmacological adjuvant agents, promoting or concurring to the therapeutic effect, such as steroidal hormones, peptide hormones and growth factors (GH, IGF I), substrates, enzymes, extracts, electrolytes, drugs etc.

A further object of the invention is a method for the activation of processes of specific differentiation of stem cells in vitro, characterized by the fact that stem cells and mature cells of adult tissue are placed in a culture medium and an effective quantity of relaxin is added to the culture as pro-differentiating agent for promoting the differentiation of the stem cells towards the phenotype represented by the said mature cells. The method can be applied to stem cells of various type, especially nervous or cardiac, both on totipotent and pluri- or multipotent cells of various origin.

A further object of the present invention is a medicament for the reconstructive treatment of damaged tissues, characterized in that it contains a therapeutically acceptable quantity of relaxin or of one of its pharmacologically acceptable derivatives.

An object of the invention is also a therapeutic method based on the use of relaxin in combination with stem cells. The method can envisage the combined administration, especially by injection, of stem cells and relaxin. Administration can take place by direct injection in the tissues to be reconstructed, for example by intratissue injection or (in the case of reconstruction of the tissue of the myocardium) by intracoronary injection. However, administration of relaxin and the stem cells in various zones of the patient's body and allowing them to migrate spontaneously to the damaged tissue is not excluded.

Further specific aspects of the invention are indicated in the appended claims.

### BRIEF DESCRIPTION OF THE FIGURES

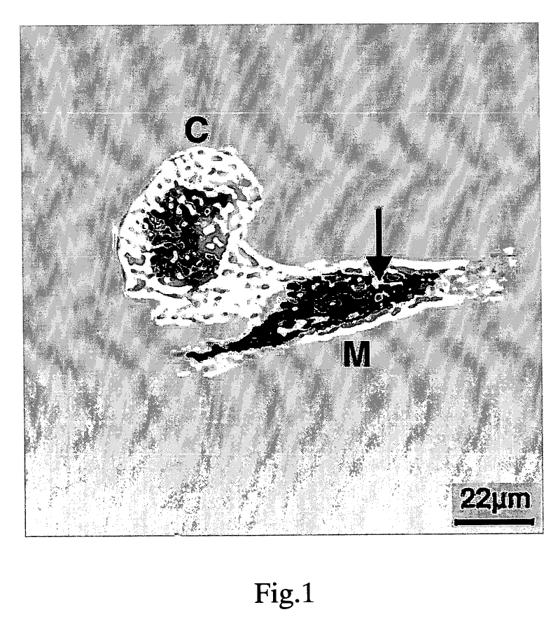

The appended FIGS. 1 to 4 show the experimental results obtained by the use of relaxin in co-cultures of myoblasts and cardiomyocytes.

### DETAILED DESCRIPTION OF EXPERIMENTAL APPLICATIONS

Use of RLX as Adjuvant in the Differentiation of Cardiac Stem Cells: Interaction Between Cardiomyocytes and Skeletal Myoblasts in Culture In Vitro

Myoblasts of mouse of the strain C2C12 obtained from the American Type Culture Collection (ATCC, Manassas, USA) were cultivated in DMEM medium containing 10% of fetal bovine serum and 0.1% of gentamicin, in an atmosphere containing air at 95% and CO₂ at 5% and at a temperature of 37° C. The medium was changed every 48 hours. Once a confluence of 90% had been reached, the cells were induced to differentiate by cultivating them in DMEM medium without serum with 0.1% of bovine serum albumin (BSA) for 24 hours.

Primary cultures of rat cardiomyocytes were obtained from hearts of male rats of the Wistar strain of about 250-300 g body weight by enzymatic digestion of the tissue with collagenase 1, according to a known method described for example in Nistri S., Mazzetti L., Failli P. and Bani D. High-yield method for isolation and culture of endothelial cells from rat coronary blood vessels suitable for analysis of intracellular calcium and nitric oxide biosynthetic Pathway, BPO (Biological Procedures Online), 2002), 4: 32-37.

On the very day of isolation, the rat cardiomyocytes were put together with the skeletal myoblasts C2C12 in ratio of 1:3. The co-cultures thus obtained were plated on cover-glasses coated with laminin and placed in the wells of a 6-well multiple plate for cellular cultures at a total cellular density of 10⁵ cells per well. The co-cultures were maintained in medium M199 with addition of L-carnitine (2 mM), creatine (5 mM), taurine (5 mM), albumin (0.2%), 15% of fetal bovine serum for 24, 48, 72 and 96 hours. In a similar group of experiments, human recombinant RLX was added to the co-cultures (10 nmol/l).

The results described hereunder obtained with human recombinant relaxin are also obtained with purified natural porcine relaxin.

Said concentration was chosen as it had proved effective for inducing an evident biological effect in previous studies conducted on cells in culture. The cellular cultures were examined with an inverted optical microscope (Nikon) equipped with a CCD video camera.

In particular, an assay of intercellular transfer of Lucifer yellow was carried out. The dye Lucifer yellow (Molecular Probes, Eugene, USA) was used since it has a molecular dimension compatible with the channels of the connexons of the gap junctions. A solution of Lucifer yellow (0.5 mg/ml in PBS) was inserted into individual cells using a micro-injection apparatus under a phase contrast microscope (Eppendorf) (180 kPa of inflow pressure for 0.4 second). The C2C12 skeletal myoblasts were injected when they were juxtaposed With the cardiomyocytes and vice versa. Functional coupling between the two cellular types was evaluated by means of a system of fluorescence videomicroscopy recording the transfer of the fluorescence of the Lucifer yellow, registered by setting the equipment to an excitation wavelength of 488 nm and for recording an emission wavelength of 512 nm. The specificity of transfer of the dye across the gap junctions was verified by pretreating the cellular cultures with ethanol (3.5 mM), a reversible blocker of the connexons.

To verify the possibility of a functional coupling that involves the passage of Ca²⁺ between the correlated cells, the co-cultures of C2C12 myoblasts and rat cardiomyocytes were treated with the fluorochrome sensitive to Ca²⁺, Fluo-3 AM (Molecular Probes) for 20 minutes at 37° C., as described in previous works (see Formigli L., Francini F., Meacci E., Vassalli M., Nosi D., Quercioli F., Tiribilli B., Bencini C., Piperio C., Bruni P. and Zecchi-Orlandini S. (2002) Sphingosine 1-phosphate induces Ca²⁺ transients and cytoskeletal rearrangement in C2C12 myoblastic cells, in Am. J. Physiol. Cell. Physiol. 282: C1361-C1373). The cover-glasses with the cells adhering on top were then transferred into suitable humid chambers mounted on a laser scanning confocal microscope Bio-Rad MCR 1024 ES (Bio-Rad, Hercules, Calif.), equipped with a krypton/argon laser source at 15 mW for observations in fluorescence provided with an immersion objective Nikon PlanApo at 60 magnification.

The co-cultures were then stimulated with specific agonists of the cardiomyocytes, such as caffeine or isoproterenol, capable of inducing selectively a mobilization of the intracellular Ca²⁺ in said cells. Individual cardiomyocytes positioned at myoblasts were also stimulated with mechanical impulses using the cantilever of an atomic force microscope (AFM, Pico SPM, Molecular Imaging, Phoenix, USA), capable of exerting a depression on the plasma membrane of 2 μm for 0.5 second at the frequency of 1 mechanical impulse per second.

The fluorescence of the Fluo-3 activated by bonding with the Ca²⁺ was stimulated with a laser source at excitation wavelength of 488 nm and was detected by means of a photomultiplier with an emission filter at 510 nm of wavelength. Real-time analysis of the transients of the Ca²⁺ following stimulation of the cardiomyocytes was effected with the program Time Course Kinetic (Bio-Rad). Digital images (515×512 pixels) were obtained every 0.32 second. The changes in fluorescence of the Ca²⁺ were measured in the individual cells by means of suitable software. The results were reported as a percentage relative to the fluorescence values of the cells in basal conditions. To determine the cellular contraction, together with the images in fluorescence for the Fluo-3 AM, images were recorded simultaneously in differential contrast of interference (CDI) for evaluating the variations of the form of the cells.

Confocal Immunofluorescence

Mixed cultures of C2C12 cells and rat cardiomyocytes were cultivated on cover-glasses coated with laminin, fixed in paraformaldehyde buffered at 2%, permeabilized with cold acetone and then incubated in a blocking buffer (PBS containing glycerol at 3% and BSA at 0.5%) to remove the possible nonspecific binding sites. In a series of experiments, the C2C12 cells were cultivated on their own and used as controls. To detect the expression of connexin 43 (Cx43), the cultures were incubated with mouse anti-Cx43 monoclonal antibodies (Molecular Probes; dilution for use 1:200) for 1 hour at room temperature and the immunoreaction was then detected by means of goat anti-mouse IgG conjugated with fluorochrome Alexa-488 (Molecular Probes). For immunodetection of myogenin, a marker of myoblastic differentiation in the skeletal direction, the cells were treated with anti-myogenin monoclonal antibodies (Santa Cruz; dilution for use 1:100) for 1 hour at room temperature and the immunoreaction was then detected by means of goat anti-mouse IgG conjugated with the fluorochrome Texas Red (Molecular Probes): in these cases, for better definition of the cells, they were also stained with falloidin marked with Alexa 488 (Molecular Probes) for revealing the actinic cytoskeleton. The glasses with the immunolabeled cells were then mounted in a suitable balsam for observations in fluorescence. Negative controls were obtained by replacing the aforesaid primary antibodies with non-immune mouse serum. The samples were then examined with the laser scanning confocal microscope (Bio-Rad), executing a series of optical sections at intervals of 0.4 μm.

Results: Skeletal Myoblasts and Cardiomyocvtes in Co-Culture

Investigation by confocal microscopy showed that the C2C12 murine skeletal myoblasts express the protein Cx43, marker of the gap junctions, localized primarily at the mutual points of contact and with the rat cardiomyocytes present in the co-cultures.

The junctions already became visible at 24 hours of culture and were found to be fully functioning: in fact, the Lucifer Yellow dye microinjected in the myoblasts was easily transferred to the adjacent cardiomyocytes. The points at which the microinjections took place are indicated by the arrows in FIG. 1. In that figure the junctions are demonstrated by the passage of Lucifer Yellow between a myoblast M and a cardiomyocyte C. The image has been processed to make the part stained with Lucifer Yellow darker and make it more visible in black and white reproduction.

Said phenomenon was inhibited by heptanol, a specific blocker of the gap junctions.

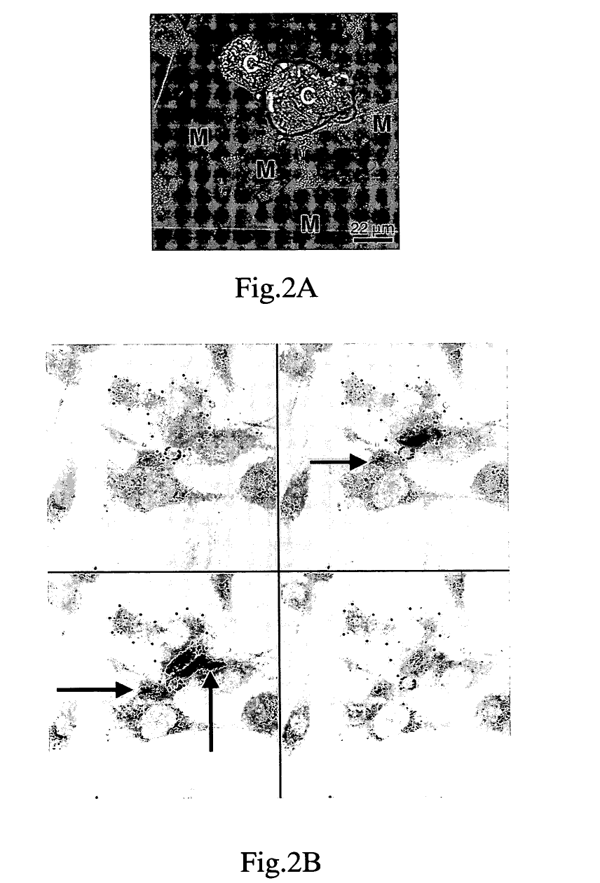

The presence of functionally active gap junctions is known to be responsible for the electrical coupling between cardiac cells, the process that triggers the coordinated contraction of the myocardium: in the present research, the experiments with the calcium-sensitive fluorochrome Fluo-3 AM showed how stimulation of the cardiomyocytes, both with specific agonists such as caffeine and isoproterenol and with mechanical impulses applied to the plasmalemma, causes a rapid increase of intracellular Ca²⁺, which spreads all of a sudden to the adjacent myoblasts, a clear sign that the gap junctions are able to permit functional coupling between the two cytotypes in co-culture. FIGS. 2A and 2B again indicate with C and M the cardiomyocytes and the C2C23 myoblasts respectively. The co-cultures were submitted to loading with Fluo-3AM. The arrows indicate the propagation from the cardiomyocytes to the adjacent myoblasts of a calcium transient obtained by stimulation of the cardiomyocytes by means of caffeine. In FIG. 2B the outline of the cardiomyocytes is marked with a dotted line.

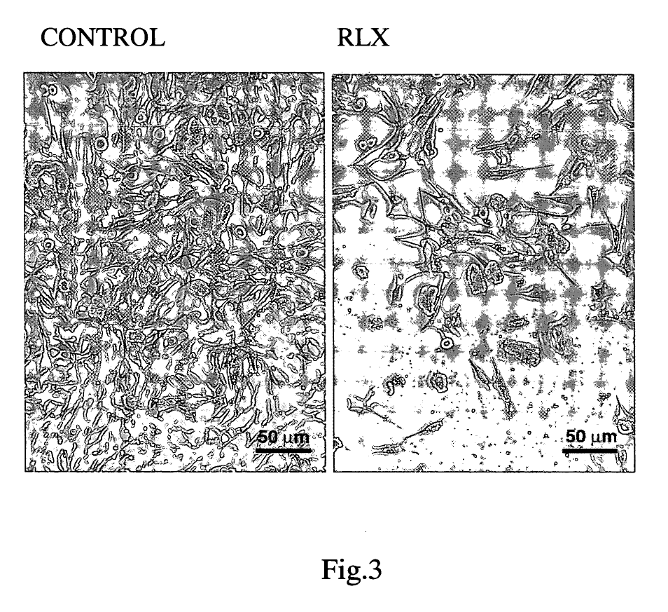

Incubation of the co-cultures with RLX (10 nmol/l) caused an appreciable reduction in the proliferative rhythm of the C2C12 myoblasts, slowing their rate of differentiation in myotubes and promoting in this way their interconnection with the cardiomyocytes. FIG. 3 shows on the left a co-culture of C2C12 myoblasts and control cardiomyocytes and on the right a co-culture of C2C12 myoblasts and cardiomyocytes treated with RLX in the culture medium. After 72 hours of incubation, the number of myoblasts is appreciably lower in the co-culture treated with RLX than to the control culture.

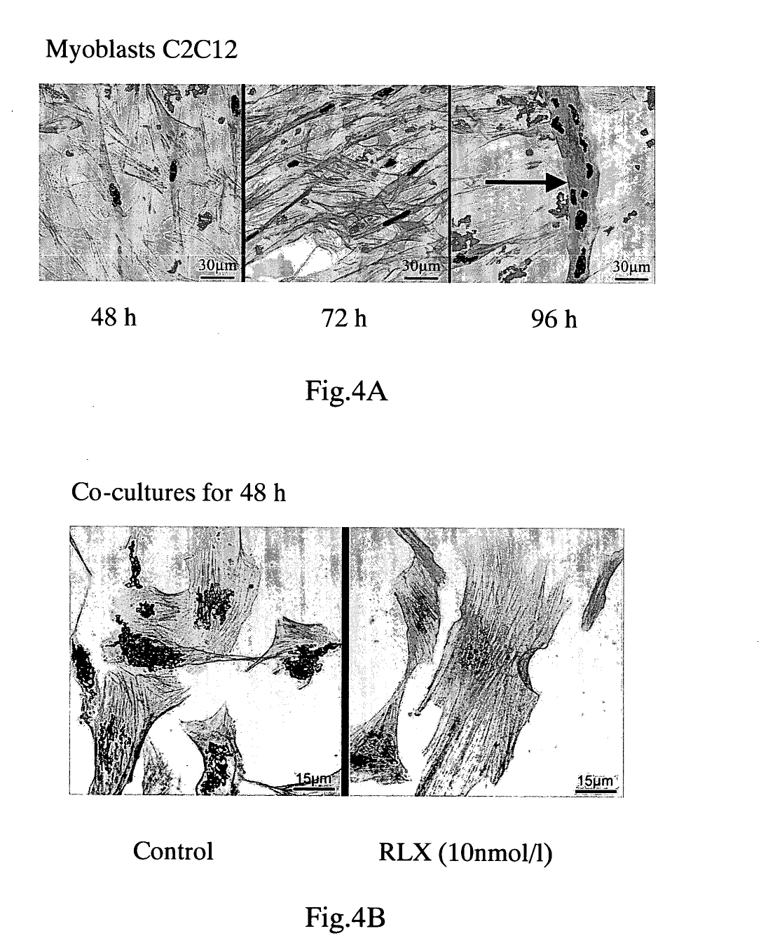

The RLX caused moreover a decrease depending on the time of expression of the nuclear protein myogenin, considered a marker of differentiation in the skeletal direction of the myoblasts which start to form the myotubes, syncytial precursors of the skeletal striated muscle fibers. This effect can be seen in FIGS. 4A and 4B. FIG. 4A shows a culture of C2C12 myoblasts in the absence of RLX. In the temporal progression of the photographs, taken at intervals of 48, 72 and 96 hours, it can be seen that the nuclear myogenin increases with the culture time. At 96 hours the presence of a myotube is noted (arrow). FIG. 4B shows the co-culture of C2C12 myoblasts and control cardiomyocytes (photo on the left) and with RLX. The expression of myogenin is maintained in the control and appears reduced after treatment with RLX. In the microphotographs the actinic filaments of the cytoskeleton can also be observed.

This indicates that RLX is able to obstruct the differentiation of the myoblasts in skeletal direction, thus promoting functional coupling between these and the cardiomyocytes. It is noteworthy that both in the presence and in the absence of RLX in the culture medium, in the entire period of co-culture analyzed, the skeletal myoblasts always appeared as mononucleated cells of elongated or pavement form and were never observed to differentiate in myotubes (FIG. 4B). However, if left to grow in single culture in the absence of cardiomyocytes and/or RLX (FIG. 4A), the myoblasts tended to differentiate spontaneously in skeletal direction, expressing high levels of myogenin and uniting to form myotubes.

These interesting results obtained in vitro support the hypothesis that RLX can exert an important contribution also in the therapy of the consequences in medium and long term of cardiac infarction, facilitating the success of the techniques of CCM for the regeneration of the infarcted ventricular tissue and for the possible functional improvement of the cardiac contractility.

Use of RLX as Adjuvant in the Differentiation of Nervous Stem Cells:

The neuroblasts are physiologically present, though in scant number, in certain compartments of the central and peripheral nervous system. Also in this case their identification has suggested that it might be possible to use them for reintegrating the nerve cells lost as a result of neurodegenerative diseases, such as for example Parkinson's disease. In this case, the possible clinical application is currently still more remote than that with the myoblasts in myocardial infarction, because the neurons are cells that differentiate early during intrauterine development developing myriads of reciprocal synaptic connections that can be reproduced difficultly in a second moment. Still more crucial is therefore the necessity of identifying factors that might be able to reproduce the conditions existing during prenatal life, promoting the functional integration of putative neuroblasts “of substitution” in the damaged nervous tissue.

In experiments conducted in this direction, it was found that the addition of RLX (10-100 ng/ml) for 48-72 hours to cultures of human olfactory neuroblasts promotes the expression of nestin, marker protein of differentiation, and the appearance of typical cellular prolongations, prerequisite for the establishing of interneuronal synaptic connections, fundamental processes of neuronal maturation/differentiation.

Relaxin, therefore, can find application in the treatments of degenerative or traumatic lesions of the nervous tissue. In an exemplary, but non-limitative way, relaxin can be used in the treatment of infarction lesions, i.e. of the damage to the nerve cells deriving from the absence of blood supply on account of ictus, in the treatment of lesions consequent to traumas or losses of tissue and in the treatments of the neurodegenerative diseases, such as Alzheimer's disease and Parkinson's disease.

Moreover, the effect as agent of stimulation and pro-differentiating on the stem cells of relaxin suggests a use of it also as stimulator in syndromes deriving from missing activation of neuroblasts or of neurocytes. Typically, relaxin can find use as stimulator of the activation and of the Migration of the olfactory neuroblasts for the therapy of Kalmann's syndrome (hypogonadism with anosmia) and of other pathological conditions deriving from the missing activation of particular stem cells. More generally, the use of relaxin can be useful for the treatment of all those syndromes deriving from the missing activation of stem cells during fetal development (at subsequent somatic-functional maturation).

1. Use of for the production of a drug having a pro-differentiating effect on stem cells.

2. Use of relaxin for the production of a drug for the treatment of a condition that requires the activation of stem cells or their differentiation towards a specific phenotype for integrating a damaged tissue.

3. Use as in claim 1, for the production of a drug having a pro-differentiating effect on multipotent, pluripotent or totipotent stem cells.

4. Use as in claim 2, for the production of a drug having a pro-differentiating effect on multipotent, pluripotent or totipotent stem cells.

5. Use as in claim 1, in which said stem cells are cardiac stem cells.

6. Use as in claim 1, in which said stem cells are myoblasts or fetal cardiomyocytes.

7. Use as in claim 1, in which said stem cells are nervous stem cells.

8. Use as in claim 7, in which said stem cells are neuroblasts.

9. Use as in claim 1 for the treatment of one or more of the following lesions:
a. infarction of the myocardium
b. decompensation of the myocardium
c. muscle-tendon lesions
d. osteo-cartilagineous lesions
e. lesions of joint capsule, ligaments and tendons
f. neurological degenerative lesions, infarction-traumatic lesions of the brain, of the spinal cord, or of the peripheral nerves.

10. Use of relaxin as pro-differentiating agent on stem cells.

11. Use as in claim 10, in which said stem cells are multipotent, pluripotent or totipotent stem cells.

12. Use as in claim 9, in which said stem cells are cardiac cells.

13. Use as in claim 10, in which said stem cells are cardiac cells.

14. Use as in claim 11, in which said stem cells are myoblasts or fetal cardiomyocytes.

15. Use as in claim 10, in which said stem cells are nerve cells.

16. Use as in claim 15, in which said stem cells are neuroblasts.

17. Use as in claim 10, in which said cells are mesenchymal blastic cells.

18. Method for the activation of processes of specific differentiation of stem cells in vitro, including the steps of placing in a culture medium stem cells and mature cells of adult tissue and adding to the culture an effective quantity of relaxin as pro-differentiating agent for promoting the differentiation of the stem cells towards the phenotype represented by said mature cells.

19. Method as in claim 18, in which said stem cells are multipotent, pluripotent or totipotent stem cells of various origin.

20. Method as in claim 18, in which said stem cells are cardiac cells or said mature cells are adult cardiomyocytes.

21. Method as in claim 19, in which said stem cells are cardiac cells or said mature cells are adult cardiomyocytes.

22. Method as in claim 19, in which said stem cells are myoblasts or fetal cardiomyocytes.

23. Method as in claim 20, in which said stem cells are myoblasts or fetal cardiomyocytes.

24. Method as in claim 18, in which said stem cells are nerve cells and said mature cells are mature nerve cells.

25. Method as in claim 20, in which said stem cells are nerve cells and said mature cells are mature nerve cells.

26. Method as in claim 24, in which said stem cells are neuroblasts.

27. Method as in claim 18, in which said stem cells are mesenchymal blastic cells and said mature cells are cells of the osseous and/or cartilagineous tissue.

28. A medicament for the reconstructive treatment of damaged tissues or for the activation of stem cells, containing a therapeutically effective quantity of relaxin.

29. Medicament as in claim 28, for the treatment of infarction of the myocardium.

30. Medicament as in claim 28, for the therapy of cellular cardiomyoplasty.

31. Medicament as in claim 28, for the treatment of lesions of the nervous system.

32. Medicament as in claim 31, for the treatment of lesions deriving from neurodegenerative processes of the nervous tissue.

33. Medicament as in claim 31, for the treatment of neurological degenerative infarction or infarction-traumatic lesions, of the nervous tissue, and especially of the brain, of the spinal cord or of the peripheral nerves.

34. Medicament as in claim 28, in which relaxin is contained in an injectable vehicle.

35. Medicament as in claim 28, for the treatment of syndromes deriving from the missing activation of stem cells during fetal development.

36. Medicament as in claim 30 for the treatment of hypogonotropic hypogonadism with anosmia (Kallman's syndrome).

37. Medicament as in claim 28, for the treatment of lesions of the osteo-cartilagineous tissue.

38. Medicament as in claim 28, for the treatment of muscle-tendon lesions.

39. Medicament as in claim 28, for the tissue reconstruction of an organ.

40. Medicament as in claim 39, in which said organ is the liver.

41. Method for the reconstructive treatment of a damaged tissue by means of application of stem cells, in which a patient who requires said treatment is administered a therapeutically effective quantity of relaxin for stimulating the development and the differentiation of stem cells towards mature cells of the phenotype corresponding to the tissue to be reconstructed.

42. Method as in claim 41, in which said relaxin is administered by means of injection.

43. Method as in claim 41, in which said relaxin is administered in combination with said stem cells.

44. Method as in claim 41, in which said damaged tissue is cardiac tissue.

45. Method as in claim 44, in which said stem cells are cardiac stem cells.

46. Method as in claim 45, in which said stem cells are fetal cardiomyocytes or myoblasts.

47. Method as in claim 41, in which said damaged tissue is nervous tissue.

48. Method as in claim 47, in which said stem cells are nervous stem cells.

49. Method as in claim 48, in which said stem cells are neuroblasts.

50. Method as in claim 41, for the reconstruction of muscle-tendon tissue.

51. Method as in claim 41, for the reconstruction of osteo-cartilagineous tissue.

52. Method as in claim 41, for the reconstruction of hepatic tissue.

53. Method for the treatment of syndromes deriving from the missing activation of stem cells during fetal development, in which a patient who requires said treatment is administered a therapeutically effective quantity of relaxin to stimulate the development of stem cells.

54. Method as in claim 53, in which said syndrome is hypogonotropic hypogonadism with anosmia (Kallman's syndrome), the relaxin stimulating the activation and the migration of the olfactory neuroblasts.

55. Method for the treatment of syndromes deriving from the missing activation of neuroblasts or neurocytes, or from the lack or from the loss of neurons, in which a patient who requires said treatment is administered a therapeutically effective quantity of relaxin, for stimulating the activation of said neuroblasts or for stimulating the differentiation of said neuroblasts in mature nerve cells.
